SEQUENCE LISTING (8 sequences)

SEQ ID NO: 1 - DNA, 36 bp, Artificial Sequence
  Primer
tattatgagctcggtgctgtgaccgtttccaatacg

SEQ ID NO: 2 - DNA, 44 bp, Artificial Sequence
  Primer
tattatactagttggtatttctgatgatgttcttgctctctttg

SEQ ID NO: 3 - DNA, 40 bp, Artificial Sequence
  Primer
tattatactagtatgagatttccttcaatttttactgcag

SEQ ID NO: 4 - DNA, 33 bp, Artificial Sequence
  Primer
tattatgtcgacttagtacattggttggccggg

SEQ ID NO: 5 - DNA, 70 bp, Artificial Sequence
  Primer
aagaagattacaaactatcaatttcatacacaatataaacgattaaaagacggatccccgggttaattaa

SEQ ID NO: 6 - DNA, 70 bp, Artificial Sequence
  Primer
tgggaacaaagtcgactttgttacatctacactgttgttatcagtcgggcgaattcgagctcgtttaaac

SEQ ID NO: 7 - DNA, 70 bp, Artificial Sequence
  Primer
ttactaccatcacctgcatcaaattccagtaaattcacatattggagaaacggatccccgggttaattaa

SEQ ID NO: 8 - DNA, 70 bp, Artificial Sequence
  Primer
atgaacgtgaaagaaatcgagagggtttagaagtagtttagggtcattttgaattcgagctcgtttaaac

(19) United States
(12) Patent Application Publication
(10) Pub. No.: US 2010/0285505 A1
(43) Pub. Date: Nov. 11, 2010

(54) Device and Method for Detecting a Substance by Means of Particle Plasmon Resonance (PPR) or Particle-Mediated Fluorescence Based on Cell Surface Polarizations

(75) Inventors: Kai Ostermann, Dresden (DE); Wolfgang Pompe, Hartha (DE); Dagmar Wersing, Dresden (DE); Mathias Lakatos, Dresden (DE); Michael Mertig, Dresden (DE); Gerhard Rödel, Karlsfeld (DE); Simone Thierfelder, Dresden (DE)

Correspondence Address: GUDRUN E. HUCKETT DRAUDT, SCHUBERTSTR. 15A, WUPPERTAL, 42289, DE

(73) Assignee: TECHNISCHE UNIVERSITAET DRESDEN, Dresden (DE)

(21) Appl. No.: 12/666,299

(22) PCT Filed: Jun. 27, 2008
(86) PCT No.: PCT/EP2008/058288

§ 371 Date: Jun. 19, 2010

(30) Foreign Application Priority Data
Jun. 27, 2007 (DE) 10 2007 031 533.5

(51) Int. Cl. G01N 33/53; C12M 1/34

(52) U.S. Cl. 435/7.31; 435/287.2

(57) ABSTRACT

The invention relates to devices and methods for detecting a substance by cell surface polarizations and the detection thereof by means of PPR or particle-mediated fluorescence. The device for detecting a substance by cell surface polarization according to the invention has cells, wherein a gene the expression of which leads to the polarized presentation of a protein on the surface of the cell is placed under the control of a promotor which can be regulated by the substance to be detected, nanoparticles which are functionalized with a molecule which can bind specifically to the surface-exposed protein, and at least one optical measurement device, such that an accumulation of the nanoparticles on the surface of the cells can be detected by particle plasmon resonance or particle-mediated fluorescence.

The invention relates to devices and methods for detecting a substance by means of cell surface polarization and its detection by means of PPR or particle-mediated fluorescence.

Known systems do not allow for signals detected by living cells to be converted such that they can be detected in a simple way by means of PPR or particle-mediated fluorescence in the immediate neighborhood of the living cells.

It is an object of the invention to provide a device and a method for detection of a substance by means of a biological system by utilizing particle plasmon resonance (PPR) or particle-mediated fluorescence with which target molecules, possibly even in minimal concentrations, can be detected.

According to the invention, the object is solved by a device for detection of a substance by cell surface polarization with the features of claim 1, that is:
a) cells in which a gene, whose expression leads to the polarized presentation of a protein on the surface of cells, is under the control of a promoter that can be regulated by the substance to be detected,
b) nanoparticles that are functionalized with a molecule that binds specifically to the surface-exposed protein,
c) at least one optical measuring device,

so that by particle plasmon resonance or particle-mediated fluorescence an aggregation of nanoparticles on the surface of the cells can be detected.

A typical example of the polarization of surfaces is the formation of the immunological synapsis. When, for example, human T-cells are activated by the presentation of an antigen, a redistribution of the T-cell receptors that had been previously ubiquitously distributed on the cell surface occurs toward the point of antigen recognition. By redistribution a strong accumulation of the T-cell receptors in a limited area of the cell surface results, the so-called immunological synapsis (van der Merwe, P. A. et al. (2000) Cytoskeletal polarization and redistribution of cell-surface molecules during T-cell antigen recognition. Seminars in Immunology 12:5-21).

The activations of, for example, CD4 and CTLA-4 receptors, also lead to polarization effects in case of T-cells (Nguyen, D. H. et al. (2005) Dynamic reorganization of chemokine receptors, cholesterol, lipid rafts, and adhesion molecules to sites of CD4 engagement. Experimental Cellular Research 304:559-569; Wei, B. et al. (2007) CTL-associated antigen-4 ligation induces rapid T-cell polarization that depends on phosphatidylinositol 3-kinase, Vav-1, Cdc42, and myosin light chain kinase. The Journal of Immunology 179:400-408).

A further example for a high level polarization of a biomolecule on cell surfaces after specific induction is exhibited by Dictyostelium discoideum. This is a single-cell slime mold that may exist as an amoeba or, after induction, organized as a cell aggregate up to a multi-cell sporocarp. The aggregation of the individual cells is controlled by chemotaxis and requires a high-level regulated genetic program which in the end leads to formation of a sporocarp. When D. discoideum is exposed to a chemical gradient (e.g.. cAMP), polarization of the cell surface takes place. A typical marker of this polarization is the phospholipid PIP3 that on the cell surface accumulates specifically toward the point of highest concentration of the gradient. The accumulation of PIP3 is observed in many chemotactic cells (Franca-Koh, J. et al. (2006) Navigating signaling networks: chemotaxis in Dictyostelium disoideum. Current Opinion in Genetics and Development 16:333-338).

A further example of a protein that after induction exits as a cluster on the cell surface is chemokine receptor CCR2. CCR2 is a membrane-bound G protein-coupled receptor that projects from the cell. It is activated by chemokines and transmits the signal through the plasma membrane which leads to the activation of a downstream signal cascade. CCR2 is presented in polarized form in response to specific stimuli, for example, in lymphocytes or also in multipotent adult mesenchymal stem cells, on the cell surface

(Belema-Bedada, F. et al. (2008) Efficient homing of multipotent adult mesenchymal stem cells depends on FROUNT-mediated clustering of CCR2. Cell Stem Cell 2:566-575; Meto, M. et al. (1997) Polarization of chemokine receptors to the leading edge during lymphocyte chemotaxis. Journal of Experimental Medicine 186:153-158).

In a preferred embodiment, the expression of the gene in a first cell induced by the substance to be detected causes a polarized presentation of a protein on the surface of the cells of a second cell type. The corresponding configuration therefore has the features of claim 2, i.e.,
cells of a first type in which a gene that codes for a pheromone is under the control of a promoter that can be regulated by the substance to be detected so that the presence of the substance to be detected causes the cells of the first type to secrete the pheromone, and
cells of a second type that are-responsive to the pheromone and whose surface is polarized by the presence of the pheromone.

The device according to claim 2 can therefore be summarized as follows as a device with:
cells of a first type in which a gene that codes for a pheromone is under the control of a promoter that can be regulated by the substance to be detected so that the presence of the substance to be detected causes the cells of the first type to secrete the pheromone, and
cells of a second type that are responsive to the pheromone and whose surface is polarized by the presence of the pheromone,
nanoparticles that are functionalized with an antibody that is directed against a protein that is exposed in polarized form on the surface of the cells of the second type by the presence of the pheromone, and
at least one optical measuring device,

so that a measurable aggregation of the nanoparticles on the cell surface of the yeast cells of the second type can be detected by particle plasmon resonance or optical fluorescence.

In the device according to the invention the cells are present in a liquid, preferably an aqueous medium. The cells of the device are either suspended in solution or immobilized on a carrier.

The solution is disposed in a suitable container that ensures the measuring-technological detection of a signal emitted by the cells.

The carrier is also designed such that signals emitted by the cells can be detected by a detection system.

The substances to be detected are also in aqueous solution. For the detection the solutions to be tested are contacted with the cells of the device according to the invention.

Advantageous embodiments of the device according to the invention are disclosed in the claims 3 to 29.

According to the embodiment of claim 3, the cells are yeast cells. According to the embodiment of claim 4, the cells are Saccharomyces cerevisiae or Schizosaccharomyces pombe yeast cells

Yeast cells are characterized by two so-called mating types. In case of baker's yeast Saccharomyces cerevisiae these are the mating types a and a; in case of fission yeast Schizosaccharomyces pombe plus and minus.

The yeast cells of the first type according to the embodiment of claim 5 are Saccharomyces cerevisiae cells of the mating type a or Saccharomyces cerevisiae cells of the mating type a.

The yeast cells of the second type according to the embodiment of claim 6 are Saccharomyces cerevisiae cells of the mating type a or Saccharomyces cerevisiae cells of the mating type α. The yeast cells of the second type have receptors as well as a correlated intracellular signal cascade for transmitting the signal for the pheromone that is produced by the cells of the first type.

The respective yeast cells form short peptides, so-called pheromones, in order to communicate to their environment the their own mating type. For example, Saccharomyces cerevisiae cells of the mating type a secrete the pheromone α-factor and cells of the mating type a secrete the pheromone a-factor. The yeast cells have on their surfaces receptors for the pheromones of the respective opposite mating type. For example, Saccharomyces cerevisiae cells of the mating type a are capable of recognizing Saccharomyces cerevisiae cells of the mating type α in their environment and vice versa.

When yeast cells of one mating type recognize pheromones of the opposite mating type in their environment, a complex genetic program is started whose goal is mating of one yeast cell of one mating type with one yeast cell of the opposite mating type, respectively, with formation of a diploid zygote.

According to the invention, yeast cells of a first type are genetically modified such that a gene that codes for a pheromone is under the control of a promoter that is regulated by a signal.

A promoter is defined in genetics as a DNA sequence that regulates the expression of a gene. Promoters in the context of the invention are preferably those segments of the genomic DNA that are specifically responsible for the regulation of the expression of a gene in that they react to specific intracellular or extracellular signals and, depending on these signals, activate or repress the expression of the gene under their control. In yeasts these regulating DNA segments are in general at the 5′ end of the start codon of the respective gene and have an average length of 309 by (Mewes H. W. et al., Overview of the yeast genome. Nature (1997) 387, 7-65). Such regulating segments may also be father removed than 1,000 by from the coding sequence or at the 3′ end of the coding sequence of the respective gene or even within the transcribed sequence of the respective gene. When such promoters are positioned at the 5′ end of the start codon of any gene, preferably a pheromone gene, they regulate the activity of this gene as a function of the aforementioned specific signals.

The gene that codes for a pheromone is according to the embodiment of claim 7 the MFα1 gene, the MFα2 gene, the MFA1 gene or the MFA2 gene.

The pheromone gene that is under the control of a promoter of a gene that is regulated by a specific signal is introduced into a yeast cell. It may be present within the yeast cell on an extrachromosomal DNA molecule. Preferably used for this purpose is a yeast expression vector that upon division of the yeast cell is replicated stably.

Especially preferred is a so-called “high copy number” vector that in the yeast cell is present in a large number of copies. Alternatively, as extrachromosomal DNA molecules also yeast artificial chromosomes are used.

In another embodiment the pheromone gene together with the promoter is integrated into the chromosomal DNA of the yeast cell. In this way, it is advantageously ensured that all progeny of the yeast cell contain also the pheromone gene under the control of the specific promoter.

Alternatively, vectors are also utilized that are present in minimal copy number or as an individual vector in yeasts, e.g. ARS-CEN vectors.

When the yeast cells of the first type recognize by means of receptors the incoming signals, directly or indirectly by intermediately positioned signal cascades the transcription of the signal-specific promoter is induced so that the yeast cells of the first type as a response to the incoming signal secrete the pheromone into the environment.

The yeast cells of the second type have on their surface receptors for the pheromones that are secreted by the yeast cells of the first type. When the secreted pheromone reaches the surrounding yeast cells of the second type, the yeast cells of the second type are arrested under the influence of the pheromone of the first type in a certain cell cycle phase (G1), metabolic processes are modified, and the yeast cells of the first and second type grow in a targeted fashion toward another. This effect is referred to in case of S. cerevisiae also as “shmoo phenotype”. The cell growth in the direction toward the mating partner is accompanied by a high-level modification and polarization of the cell surface of the yeast cells. In particular, the cell tip of the yeast cell of the second type that grows in the direction toward the yeast cell of the first type, i.e. toward the cell secreting the pheromone, experiences complex modifications in their lipid and protein composition. For example, certain proteins are present highly specific in high concentrations.

According to the embodiment of claim 8, the cell is a haploid yeast cell that is gene-technologically modified such that the gene for a pheromone of the opposite mating type is under the control of a promoter that is regulated by the substance to be detected.

The cell is responsive to these secreted pheromones. In this way, the expression of the gene that is induced by the substance to be detected causes in this cell a polarized presentation of a protein on the surface. This can then be detected by means of attachment of the functionalized nanoparticles by means of particle plasmon resonance or optical fluorescence.

According to the embodiment of claim 9, in the yeast cells of the first and/or second type the authentic regulation of the expression of pheromones is switched off.

For example, according to the embodiment of claim 10, the natural genes MFα1 and MFα2 that both code for the α-factor are deleted in α cells of Saccharomyces cerevisiae cells. In this way, it is advantageously ensured that the α-factor is produced and secreted exclusively when the signal to be detected is present.

For example, according to the embodiment of claim 11, the natural genes MFA1 and MFA2 are deleted in a-cells of Saccharomyces cerevisiae cells. In this way, advantageously secondary effects on the a-cells or α-cells are prevented.

According to the invention, nanoparticles are functionalized with a molecule that specifically binds to a protein that is exposed in polarized form by the pheromone of the first mating type on the cell surface of the yeast cells of the second mating type. Nanoparticles are particles with a diameter of approximately 1 nm up to approximately 500 nm whose optical properties depend greatly on the particle sizes as well as the particle shape. In case of nanoparticles with a size of typically >3 nm, the optical behavior is determined by plasmon resonances while for nanoparticles with a particle size <3 nm a particle-mediated fluorescence is observed. Aggregations of the nanoparticles as a result of electromagnetic interaction cause a change of the plasmon resonances (frequency shift) or a change of the fluorescence spectrum.

In an embodiment according to claim 12 the nanoparticles are comprised of gold, silver or an alloy of these metals.

Preferred are as molecules those that bind specifically to the proteins presented in polarized form on the surface of cells of the second type. These are, for example, peptide ligands, receptors, antibodies, haptenes or nucleic acids. Preferably these molecules bind to the area of the protein that is accessible at the exterior of the cell surface.

In the presence of the nanoparticles, for example, gold nanoparticles that are functionalized with molecules that specifically bind to a protein that is presented in polarized form on the cell surface because of the pheromone, duster formation on the corresponding surface areas of the yeast cells results. The resulting frequency or color changes as a result of the particle plasmon resonance or the particle-mediated fluorescence is detected by sensors. According to the invention, by particle plasmon resonance or particle-mediated fluorescence in this way a measurable aggregation of the nanopartides on the cell surface of the cells of the second mating type can be detected. An increasing aggregation of the nanoparticles (formation of clusters on the cell surface) is reflected in a frequency change (shift to red)

The device according to the invention has the advantage that a signal that is received by a cell is converted and subsequently can be detected by means of particle plasmon resonance or particle-mediated fluorescence. The signal that is received by a cell can optionally also be amplified.

According to the embodiment of claim 13, the nanopartides have a diameter of greater than 3 nm whose attachment on cells of the second type can be detected by means of particle plasmon resonance.

The nanopartides have according to the embodiment of claim 14 a diameter of less than 3 nm whose attachment to cells of the second mating type by means of particle-mediated fluorescence can be detected.

Binding of the specifically binding molecules to the nanoparticles that, as disclosed in claim 13, have a diameter of greater than 3 nm is realized directly by means of unspecific adsorption of the molecules on the surface of the nanoparticles but also in a targeted fashion specifically in case of a preceding functionalization of the nanoparticle surface (for example, by chemically functional terminal groups).

Nanoparticles that have a diameter of smaller than 3 nm, as mentioned in claim 14, are bound to antibodies as specifically binding molecule and can moreover be functionalized by means of antibody-coupled amphiphilic core/shell structures on the basis of saccharide-functionalized dendritic polymers.

In order to facilitate accessibility of the cell surface for the functionalized nanoparticles, a yeast mutant that is without cell wall is used or the cell wall is removed by prior enzymatic decomposition. For this purpose, the cells must be embedded prior to this in an osmo-stabilizing matrix (for example, 1% agarose) or kept in an osmotically stabilizing medium (for example, in 1 M sorbitol) in order to ensure integrity of the cells.

According to the claim 15, the protein that is presented in a polarized form on the cell surface by the pheromone and to which the specific molecule with which the nanoparticles are functionalized binds is Fust1p.

Fus1 p of Saccharomyces cerevisiae is required for fusion of the cells and is therefore present in high concentrations in the cell tips that grow toward one another. It is a protein that extends through the cytoplasm membrane with a transmembrane domain. The N-terminal end of Fus1p is oriented outwardly and the larger C-terminal segment is oriented into the interior of the cell. In this way, the N-terminal segment of Fus1p as a response of the cell to the action of the respective opposite pheromone is concentrated specifically on the cell surface of the cells growing toward one another.

Preferred molecules that bind specifically to Fus1p are in this connection anti-Fus1p antibodies. In the context of the present invention, moreover different modified forms of antibodies are to be understood as antibodies, for example, fragments such as the Fv fragment, the Fab fragment or the (Fab)'2 fragment.

In addition to the indicated protein also other proteins are presented either only partially or polarized on the cell surface. Such proteins can be utilized also. Moreover, the composition of the cell wall at the “cell tips” is modified and can thus be utilized for the described method.

Moreover, preferably proteins are used that are exposed in polarized form on the cell surface by activation of the pheromone signal pathway in cells of the second type and that in the area that is accessible from the exterior of the cell are bound to an epitope tag. Such epitope tags are short molecules, mostly oligopeptides, that may be present also in multimerized form and that are specifically bound by antibodies. Preferred epitope tags are those that do not impair or negatively affect the presentation of the protein that is bound to the epitope tag. Especially preferred are HA tag, Myc tag, Flag tag, SUMO tag, His tag, or T7 tag. Preferably also proteins (for example, eGFP) or protein domains in a fusion protein can be used with the protein that is presented in polarized from on the surface that are specifically bound by antibodies or nucleic acids. As molecules that specifically bind to the proteins that are presented in polarized form on the surface of the cells of the second type antibodies are used that are specifically directed against the employed epitope tag or the fusion proteins or the protein domain or nucleic acids that bind to a DNA binding protein part. In this way, advantageously a reliable and highly specific binding of the nanoparticles to the polarized presented proteins is ensured even when an antibody that is specifically directed against this protein is not available.

According to the embodiment of claim 16 with a suitable selection of the ratio of cells of the first type to cells of the second type a signal amplification results. With a targeted influence of the numerical ratio of the different cell types the amplification effect can be further increased. For example, the cells of the first type relative to the cells of the second type are present in a ratio of 1 to 20, preferably of 1 to 10, especially preferred of 1 to 5.

The cells of the device according to the invention can be suspended in solution or can be immobilized. According to the embodiment of claim 17, the cells are present in a porous organic or inorganic gel, according to the embodiment of claim 18 in a porous and optically transparent silicon dioxide xerogel.

Xerogels are gels that have lost their liquid, for example, by evaporation or applying vacuum. Gels are shape-stable, easily deformable disperse systems of at least two components that are comprised usually of a solid material with elongate or greatly branched particles (for example, silicic acid, gelatin, collagens, polysaccharides, pectins, special polymers, for example, polyacrylates, and other gelling agents that are frequently referred to as thickening agents) and a liquid (usually water) as a dispersion medium. In this connection, the solid substance in the dispersion medium produces a three-dimensional network. When xerogels are formed, the three-dimensional arrangement changes.

The use according to the invention of inorganic or biologically inert organic xerogels for embedding the cells enables advantageously the survival of the cells while providing simultaneously stability of the produced structures because they are toxicologically and biologically inert and in general are not decomposed by the yeasts. They enable moreover advantageously the incorporation of nutrients and moisturizing agents that ensure survival of the cells.

According to the invention, the cells are immobilized in a porous and optically transparent inorganic or biologically inert organic xerogel. Preferably, the xerogel is an inorganic xerogel of silicon dioxide, alkylated silicon dioxide, titanium dioxide, aluminum oxide and their mixtures and is preferably produced by a sol-gel process.

For this purpose, first silica or other inorganic nanosols are produced either by add-Catalyzed or alkali-catalyzed hydrolysis of the corresponding silicon alkoxide or metal alkoxide in water or in a water-soluble organic solvent (such as ethanol).

Preferably, hydrolysis is carried out in water in order to prevent toxic effects of the solvent on the cells to be embedded. When producing nanosols by alkoxide hydrolysis, during the course of the reaction alcohols are produced that are subsequently evaporated from the obtained nanosols by passing through an inert gas flow and replaced by water.

By using mixtures of different alkoxides the matrix properties can be affected in a targeted fashion. The sol-gel matrix enables advantageously the chemical modification by co-hydrolysis and co-condensation by utilizing different metal oxides of metals such as Al, Ti, Zr for producing mixed oxides or of alkoxy silanes with organic residues on the Si atom for producing organically modified silicon oxide gels.

The cells to be embedded are mixed with the resulting nanosol. The process of gel formation is initiated preferably by increasing the temperature, neutralizing the pH value, concentration or addition of catalysts, for example, fluorides. However, in this connection, the temperature should not be increased to temperatures of >42° C. in order not to damage the cells to be embedded. When converting into a gel, the nanosols reduce their surface area to volume ratio by aggregation and three-dimensional cross-linking. During this conversion of the nanosol into a so-called lyogel the cells are immobilized in the resulting inorganic network. The immobilization of cells capable of survival is advantageously controlled by the ratio of cells to oxide and by addition of pore-forming agents.

The proportion of cells in the total quantity of the generated xerogel including the embedded cells, depending on the application. can be from 0.1 to 50% by weight. Preferred is a proportion of 2 to 25% by weight.

By drying, solvent that is still contained in the lyogel is removed. In this way, the gel is converted to the xerogel. The resulting xerogel has a high porosity that enables fast material exchange with the surrounding medium. The drying process causes great shrinkage of the gel that leads to stress for the embedded cells. Preferably, the drying step is therefore performed very gently and slowly at temperatures of less than 40° C.

With decreasing water content of the matrix the physiological activity and the survival rate of the embedded cells will drop. A water content that is too high leads however to low mechanical stability and reduces the durability of the structure.

The use according to the invention of yeast cells is therefore particularly advantageous because yeast cells have a high resistance with respect to dryness and even at very minimal water contents do not lose their survival capability. In this way, it is possible to produce very dry xerogels.

The invention comprises also the use of different additives such as soluble organic salts, i.e., metal salts of organic carboxylic or sulfonic acids or open-chain or cyclic ammonia salts and quaternary salts of N-heterocycles as well as low-molecular polyanions or polycations or water-soluble organic compounds such as poly carboxylic acids, urea derivatives, carbohydrates, polyols, such as glycerin, polyethylene glycol and polyvinyl alcohol, or gelatin that act as plasticizers, moisturizing agents and pore forming agents, inhibit cell lysis, and increase significantly the survival capability of the embedded cells.

According to the embodiment of claim 19, the silicon dioxide xerogel with the cells is disposed on a substrate with increased mechanical stability.

According to embodiment of claim 20, substrates are an optical fiber, glass beads, a planar glass support or other shaped bodies of glass such as hollow spheres, rods, tubes, or ceramic granules.

In this connection, the cells are positionally fixed in a porous and optically transparent inorganic xerogel, for example, a silicon dioxide xerogel. The silicon dioxide xerogel to which the microorganisms have been added is deposited as a layer onto glass beads, an optical fiber, planar glass supports or other shaped bodies such as hollow spheres, rods, tubes or ceramic granules by means of a known sol-gel process in that the nanosol/cell mixture is applied onto the substrate to be coated or the substrate is immersed into the nanosol mixture and the nanosol is subsequently transformed by drying and the thus resulting concentration of the nanosol into a xerogel. The thus obtained mechanical stability of these structures enables the introduction of the device according to the invention into a measuring system that is connected immediately connected with the reaction space (fermenter) to be examined in the sense of a near-line diagnostics.

According to the embodiment of claim 21, the cells are a component of an envelope structure that surrounds at least partially a cavity. This means that individual or several cells are encapsulated in this cavity that has a porous envelope. The micro porosity enables advantageously a material exchange with the environment.

The envelope structure according to the embodiment of claim 22 is comprised of a base member with an inner layer of a biological hydrogel and an outer layer of a porous inorganic gel wherein the layers are applied at least partially.

According to the embodiment of claim 23, the cells are embedded in a structure with a hierarchical pore structure so that in addition to the nano porosity that is typical for inorganic gels the structure is also penetrated by mesopores that are connected with one another and whose diameter typically varies between 10 to 100 pm and that enable a material exchange between the environment and the embedded cells as well as their reaction products such as enzymes. The mesopores serve at the same time as transport paths for the nanoparticles acting as physical sensors.

The cells according to the embodiment of claim 24 are located at least on one surface in a transparent measuring cell wherein the measuring cell has devices for supplying and removing a medium and is coupled to a heating device.

The cells according to the embodiment of claim 25 are a component of a solution or a gel that is located within a container as a measuring cell.

The measuring cell is a component of an optical measuring device that is furthermore comprised of a source of electromagnetic beams and either an image recording system or a photodetector. The container and the solution or the gel are comprised of materials that are transparent for the electromagnetic beams of the source.

According to the embodiment of claim 26, as an optical measuring device an image recording system is arranged as an optical system imaging the cells such that a color change or resonance frequency change of cells caused by aggregation of the nanopartides is quantitatively or quantitatively determinable as an imaging signal.

According to the embodiment of claim 27, a source of electromagnetic beams, cells, and at least one photodetector as an optical measuring device are arranged such that electromagnetic beams of the source will impinge on cells and the thus generated fluorescent light is determinable quantitatively or quantitatively as an imaging signal of the photodetector.

According to the embodiment of claim 28, a source of electromagnetic beams, cells, as well as nanoparticles and at least one photodetector as an optical measuring device are arranged such that electromagnetic beams that are excited in the nanopartides by the electromagnetic beams of the source will reach the photodetector, are imaged thereat and, as imagining signals, are qualitatively or quantitatively determinable.

According to the embodiment of claim 29, in the beam path downstream of the source of electromagnetic beams and/or in the beam path upstream of the photodetector at least one beam-influencing device, at least one beam-shaping device or at least a combination thereof is arranged.

According to the embodiment of claim 30, the photodetector is a solid-state image sensor with photoresistors, photo diodes or photo transistors and the solid-state image sensor is connected to a data processing system.

One aspect of the invention according to claim 31 is a method for detecting a substance by means of partide plasmon resonance (PPR) or partide-mediated fluorescence by cell surface polarization with utilization of cells, nanoparticles, and at least one measuring device. The method comprises the following method steps:
a) the surface of cells, in which a gene, whose expression leads to the polarized presentation of a protein on the surface of cells, is under the control of a promoter that can be regulated by the substance to be detected, is polarized by the presence of the substance,
b) nanoparticles, functionalized with a molecule that specifically binds to the protein that upon presence of the substance is expressed in polarized form on the surface of the cells, bind to the protein, and,
c) by means of at least one optical measuring device a measurable aggregation of the nanoparticles on the cell surface of the cells is detected by particle plasmon resonance or particle-mediated fluorescence.

One aspect of the invention according to claim 32 is also a method for detecting a substance by means of particle plasmon resonance (PPR) or partide-mediated fluorescence by cell surface polarization, wherein, as cells, cells of a first type and cells of a second type are utilized, wherein
a) cells of the first type, in which a gene coding for a pheromone is under the control of a promoter that can be regulated by the substance to be detected, secrete the pheromone in the presence of the substance, and
b) the surface of cells of the second type is polarized by the presence of the pheromone.

The method according to claim 32 can thus be summarized by the following method steps:
a) the cells of the first type, in which a gene coding for a pheromone is under the control of a promoter that can be regulated by the substance to be detected, secrete the pheromone in the presence of the substance,
b) the surface of cells of a second type that are responsive to the pheromone is polarized by the presence of the pheromone,
c) nanopartides, functionalized with a molecule that can bind specifically to a protein that is exposed in polarized form by the presence of the pheromone on the surface of the cells of the second type, bind to the protein, and
d) by means of at least one optical measuring device a measurable aggregation of the nanopartides on the surface of the cells of the second type is detected by particle plasmon resonance or optical fluorescence.

For advantageous embodiments of the method according to the invention, concerning the cells, the nanopartides; and the measuring device, the advantageous embodiments of the features of the device set forth in the description of the device according to the invention apply likewise. According to the embodiment of claim 33, the method is performed with at least one device with at least one feature of one of the claims 3 to 30.

Based on the following figures and embodiments the invention with be explained in more detail.

It is shown in:

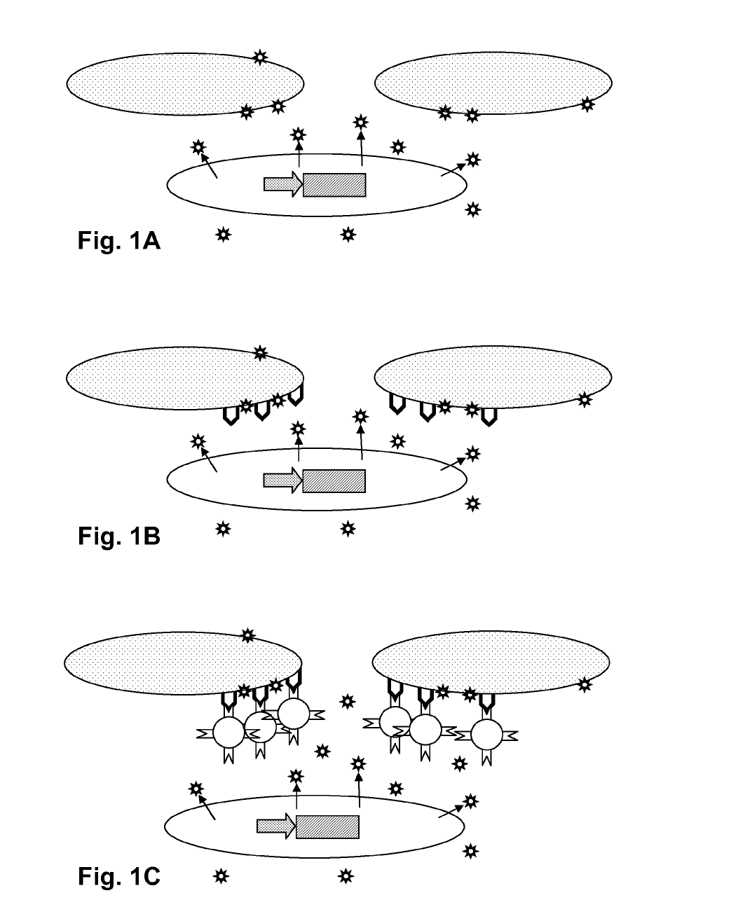

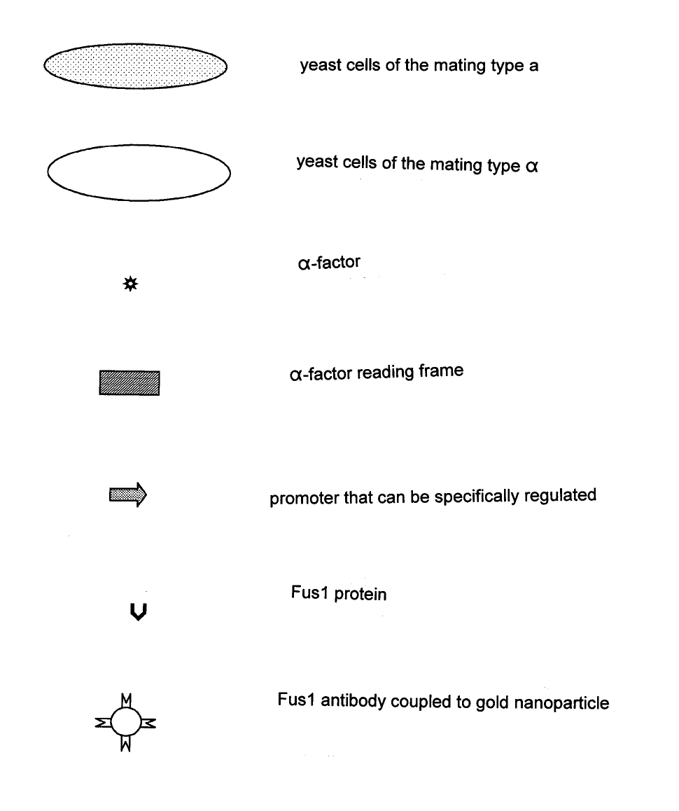

FIG. 1A a schematic representation of gene-technologically modified Saccharomyces cerevisiae yeast cells of the mating type α and of Saccharomyces cerevisiae yeast cells of the mating type a, according to Example 1;

FIG. 1B a schematic representation of the polarization of the cell surface and of clustering of the Fus1 p protein on the cell tips of the yeast cells of the mating type a, according to Example 1;

FIG. 1C a schematic representation of the gold cluster formation on the corresponding surface areas of the yeast cells of the mating type a in the presence of gold nanoparticles that are functionalized with antibodies directed against the N-terminal segment of Fus1 p or a Fus1p fusion protein oriented in the same direction, in accordance with Example 1.

### EXAMPLE 1

Saccharomyces cerevisiae yeast cells of the mating type a recognize as cells of the first type by means of a receptor an incoming signal. Receptors induce directly or by means of intermediately positioned signal cascades the transcription of the promoter. Under the control of the promoter the MFα1 reading frame coding for the α-factor is cloned so that the yeast cells of the mating type a secrete as a response to an incoming signal the pheromone α-factor into the environment.

The production of such a yeast cell of the first type is disclosed in the following in exemplary fashion for use in monitoring bio-available phosphorus. In this connection, the yeast cells of the first type (sensor cells) react sensitively to a limitation of phosphorus.

The gene YAR071W is transcribed specifically much more strongly in case of phosphorus limitation (Boer et al., (2003). The genome-wide transcriptional responses of Saccharomyces cerevisiae grown on glucose in aerobic chemostat cultures limited for carbon, nitrogen, phosphorus, or sulfur. J. Biol. Chem. 278:3265-3274.) The region of the up-regulating gene YAR071W comprising 1,000 base pairs and positioned upstream is amplified by means of the specific primer SEQ NO. 1 and SEQ NO. 2 of Table 1 by PCR of genomic DNA of Saccharomyces cerevisiae. By means of the primers, the sequence is expanded by a 5′-terminal recognition sequence for Sad and at the 3′-terminus by a recognition sequence for Spe1. By means of these recognition sequences a directed incorporation into the “high copy number” vector p426, referred to in the following by p426YAR071W, is accomplished. In the second step, the reading frame of the MFα1 gene is cloned into the plasmid p426YAR071W. For this purpose, the sequence of the MFα1 reading frame is amplified by the primers SEQ NO. 3 and SEQ NO. 4 (see Table 1) from genomic DNA of Saccharomyces cerevisiae that expand the fragment at the 5′ terminus by one Spe1 restriction site and at the 3′-terminus by one Sa/1 restriction site. Subsequently, cloning of the fragment with the aforementioned restriction sites into the vector p426YAR071W, referred to in the following as p426YAR071W-MFalpha1, is carried out. The correct sequence of the cloned fragments is checked and validated by means of DNA sequence analysis. The vector p426 contains a URA3 marker of Saccharomyces cerevisiae for selection in uracil-auxotrophic strains. The resulting construct p426YAR071W-MFalpha1 is transformed, for example, into the yeast strain BY4742 (MATα, his3Δ1, leu2Δ0, lys2Δ0, ura3Δ0) and positive transformants are selected. For phosphorus limitation, specifically the expression of the α-factor is induced in sensor cells that are provided with the plasmid p426YAR071W-MFalpha1.

TABLE 1
Primer for the production of the sensor plasmid 426YAR071W-MFalpha1.
Segments that are homolog to genomic target sequences are marked in bold,
recognition sequences for restriction endonucleases are underlined.

| No. | Name | Sequence(5′ → 3′) |
|---|---|---|
| 1 | YAR071W-for-SacI | TATTATGAGCTCGGTGCTGTGACCGTTTCCAATACG |
| 2 | YAR071W-rev-SpeI | TATTATACTAGTTGGTATTTCTGATGATGTTCTTGCTCTCTTTG |
| 3 | MFalpha1-for-SpeI | TATTATACTAGTATGAGATTTCCTTCAATTTTTACTGCAG |
| 4 | MFalpha1-rev-SalI | TATTATGTCGACTTAGTACATTGGTTGGCCGGG |

The genes MFα1 and MFα2 that authentically code for the α-factor are deleted in the same strain. In this way, it is ensured that the a factor is exclusively formed and secreted when the signal to be detected is present.

For the deletion of the reading frame of MFα1 and MFα2, for example, in the α-yeast strain BY4742 (MATα, his3Δ1, leu2Δ0, lys2Δ0, ura3Δ0) the marker cassettes natMX6 and hphMX6 are used that impart resistance against the antibiotics nourseothricin and hygromycin B. The natMX6 cassette is amplified by SFH-PCR by means of the primer SEQ NO. 5 and SEQ NO. 6 of Table 2. The 5′ end segments of the primers (50 bases each) are homolog to the adjoining sequences of the MFα1 reading frame in the genome of Saccharomyces cerevisiae. The 3′ end segments of the primers (20 bp) are homolog to the ends of the natMX6 cassette. As DNA templates for the SFH-PCR the plasmid pFA6a-natMX6 is provided. Subsequently, the yeast cells are transformed with the SFH fragment. Transformants in which the fragment is stably integrated by means of a double-homolog recombination into the genome are selected from medium containing nourseothricin and the correct integration of the deletion cassette is confirmed by means of diagnostic PCR. Subsequently, the deletion of the reading frame of MFα2 in the generated Δmfα1 yeast strain is carried out. For this purpose, in analogy to the first deletion an SHF fragment is amplified with the primers SEQ NO. 7 and SEQ NO. 8 (see Table 2) and the hphMX6 cassette (DNA template pFA6a-hphMX6) is amplified and transformed into Δmfα1 yeast cells. The 5′ end segments of the primers are homolog to the adjoining sequences of the MFα2 reading frame in the genome of Saccharomyces cerevisiae. The selection of positive transformants is realized on medium containing hygromycin B and the correct integration of the hygromycin-resistance cassette in the Δmfα1-Δmfα2 yeast strain is checked by diagnostic PCR.

TABLE 2
Primer for the deletion of the reading frame of MFα1 and MFα2 of
Saccharomyces cerevisiae. The unmarked primer sequence characterizes segments
that are homolog to the genomic DNA of Saccharomyces cerevisiae. Segments
that are homolog to the deletion cassette are marked in bold.

| SEQ | | |
|---|---|---|
| NO. | Name | Sequence (5′ → 3′) |
| 5 | MFalp1_F2 | AAGAAGATTACAAACTATCAATTTCATACACAATATAAACG |
| | | ATTAAAAGACGGATCCCCGGGTTAATTAA |
| 6 | MFalpl_R1 | TGGGAACAAAGTCGACTTTGTTACATCTACACTGTTGTTAT |
| | | CAGTCGGGCGAATTCGAGCTCGTTTAAAC |
| 7 | MFalp2_F2 | TTACTACCATCACCTGCATCAAATTCCAGTAAATTCACATA |
| | | TTGGAGAAACGGATCCCCGGGTTAATTAA |
| 8 | MFalp2_R1 | ATGAACGTGAAAGAAATCGAGAGGGTTTAGAAGTAGTTTA |
| | | GGGTCATTTTGAATTCGAGCTCGTTTAAAC |

When the α-factor that has been secreted reaches the surrounding a-cells, a high level polarization of the cell surface and clustering of the Fus1 p protein at the cell tips of the a-cells results (FIG. 1B). This clustering is the condition for the conversion into a PPR signal: The signal that is caused by the expression and secretion of the α-factor and the subsequent polarization of the cells can be modulated by the ratio of α-cells to a-cells.

In the presence of gold nanoparticles that are functionalized with antibodies that are directed against the N-terminal segment of Fus1p or a Fus1p fusion protein that is oriented in the same direction, gold cluster formation occurs at the corresponding surface areas of the a-cells (FIG. 1C). The resulting color change based on particle plasmon resonance or particle-mediated fluorescence is detected by sensors.

The source, the measuring cell and either the image recording system or the photodetector are arranged such that an optical change in the measuring cell that is caused by the electromagnetic beams of the source are imaged on the image recording system or the photodetector. The change is, for example;
a color change of yeast cells caused by the aggregation of the nanoparticles or
a fluorescent light of the yeast cells that is caused by the electromagnetic beams of the source.

The thus caused image signals of the image recording system or of the photodetector can be determined quantitatively or quantitatively. For this purpose, the image recording system or the photodetector is connected to a data processing system. In the embodiment, in the beam path downstream of the source of electromagnetic beams and/or in the beam path in front of the photodetector at least one beam-influencing device, at least one beam-shaping device or at least a combination thereof can be arranged. Beam-shaping devices are known lenses. They can expand or focus the beams so that a large surface can be exposed to beams. A beam-influencing device is preferably a scanning mirror. It is advantageously arranged and controlled such that the electromagnetic beams of the source reach as a trace the measuring cell and subsequently the image recording system or the photodetector. For several parallel traces, in the beam path in front of the scanner a pivoting mirror can be arranged. For controlling the drive of the scanner and possibly of the pivoting mirror they are connected to the data processing system. Between the source and the beam-influencing or beam-shaping device as well as between the beam-influencing or the beam-shaping device and the photodetector optical fiber devices may be arranged also so that a local separation can be provided. The image recording system is a known digital camera. The recorded digital image of the yeast cells can be processed by means of digital image processing in the data processing system and the result evaluated.

The photodetector is a solid-state image sensor with photoresistors, photo diodes or photo transistors. The resulting signals can be processed and evaluated directly by means of the data processing system.

### EXAMPLE 2

The plasmid p426YAR071W-MFalpha1 (see Example 1) for detection of phosphorus limitation is transformed directly into a strain of the mating pair a, for example, the Saccharomyces cerevisiae strain BY4741 (MATα, his3Δ1, leu2Δ0, met15Δ0, ura3Δ0).

When the formation of the α-factor is induced in this strain that has the opposite mating type, the α-factor is produced and secreted and the endogenous α-factor receptors of the strain are activated. The a-cells thus activate themselves as a result of the produced α-factor. This inter alia leads to the disclosed surface polarization and also to the “shmoo” effect.

Moreover, in the thus modified a-cells advantageously the authentic chromosomal MFα1 and MFα2 loci are transcription-inactivated.

The sensory detection of the aggregation of the nanoparticles that are functionalized with antibodies directed against the Fus1p protein is realized as disclosed in connection with Example 1.

What is claimed is:

1. Device for detection of a substance by particle plasmon resonance (PPR) or particle-mediated fluorescence by cell surface polarization, comprising:
a. cells in which a gene whose expression leads to the polarized presentation of a protein on the surface of cells is under the control of a promoter that can be regulated by the substance to be detected,
b. nanoparticles that are functionalized with a molecule that specifically binds to the surface-exposed protein,
c. at least one optical measuring device,
so that by particle-plasmon resonance or particle-mediated fluorescence a measurable aggregation of the nanoparticles on the surface of the cells can be detected.

2. Device according to claim 1, wherein the cells comprise cells of a first type and cells of a second type, wherein
a. in the cells of the first type a gene coding for a pheromone is under the control of a promoter that can be regulated by the substance to be detected so that in the presence of substance to be detected the cells of the first type secrete the pheromone; and
b. the surface of the cells of the second type that are responsive to the pheromone is polarized in the presence of the pheromone.

3. Device according to claim 1, wherein the cells are yeast cells.

4. Device according to claim 3, wherein the yeast cells are Saccharomyces cerevisiae cells or Schizosaccharomyces pombe cells.

5. Device according to claim 3, wherein the cells of the first type are Saccharomyces cerevisiae cells of the mating type a or Saccharomyces cerevisiae cells of the mating type a.

6. Device according to claim 3, wherein the cells of the second type are Saccharomyces cerevisiae cells of the mating type α or Saccharomyces cerevisiae cells of the mating type a.

7. Device according to claim 2, wherein the gene that codes for a pheromone is the MFα1 gene, the MFα2 gene, the MFA1 gene or MFA2 gene.

8. Device according to claim 3, wherein the cell is a haploid yeast cell in which the gene for a pheromone of the opposite mating type is under the control of a promoter that is regulated by the substance to be detected.

9. Device according to claim 2, wherein in the cells of the first and/or second type the authentic regulation of the expression of the pheromone is turned off.

10. Device according to claim 9, wherein the natural gene MFGα1 and MFα2 in the α-cells of Saccharomyces cerevisiae yeast cells are deleted.

11. Device according to claim 9, wherein the natural gene MFA1 and MFA2 in a-cells of Saccharomyces cerevisiae yeast cells are deleted.

12. Device according to claim 1, wherein the nanoparticles are comprised of gold, silver, or an alloy of these metals.

13. Device according to claim 1, wherein the nanoparticles have a diameter of greater than 3 nm, whose attachment on cells of the second type can be detected by means of particle plasmon resonance.

14. Device according to claim 1, wherein the nanoparticles have a diameter of smaller than 3 nm, whose attachment on cells of the second type can be detected by means of particle-mediated fluorescence.

15. Device according to claim 1, wherein the protein to which the specifically binding molecule binds is Fus1p.

16. Device according to claim 2, wherein by a suitable selection of the ratio of the cells of the first type to cells of the second type a signal amplification results.

17. Device according to claim 1, wherein the cells are disposed in a porous organic or inorganic gel.

18. Device according to claim 17, wherein the cells are disposed in a porous and optically transparent silicon dioxide xerogel.

19. Device according to claim 18, wherein the silicon dioxide xerogel with the cells is disposed on a substrate with increased mechanical stability.

20. Device according to claim 19, wherein the substrate is selected from the group consisting of an optical fiber, glass beads, a planar glass support, or-other a shaped body of glass, such as hollow spheres, rods, tubes, or and ceramic granules.

21. Device according to claim 1 one of the claims 1 to 20, wherein the cells are a component of an envelope structure that at least partially encloses a cavity.

22. Device according to claim 21, wherein the envelope structure is comprised of a base member with an inner layer of a biological hydrogel and an outer layer of a porous inorganic gel, wherein the layers are at least partially applied.

23. Device according to claim 1, wherein the cells are embedded in a structure with a hierarchical pore structure so that in addition to the nano porosity typical for inorganic gels the structure in addition also is penetrated by mesopores that are connected to one another whose diameter varies typically between 10 to 100 μm and that enable material exchange between the environment and the embedded cells as well as their reaction products such as the enzymes.

24. Device according to claim 1, wherein the cells are disposed on at least one surface in a transparent measuring cell, in that the measuring cell has devices for supplying and removing media and/or solutions and in that the measuring cell is coupled to a heating device.

25. Device according to claim 1, wherein the cells are a component of a solution or a gel that is contained in a container as a measuring cell.

26. Device according to claim 1, wherein as an optical measuring device an image recording system as an optical system imaging cells is arranged such that a color change of cells caused by aggregation of the nanoparticles can be determined quantitatively and quantitatively as an image signal.

27. Device according to claim 1, wherein a source of electromagnetic beams, cells, and at least one photodetector are arranged as an optical measuring device such that electromagnetic beams of the source impinge on cells and the thus resulting fluorescent light can be determined quantitatively or quantitatively as image signals of the photodetector.

28. Device according to claim 1, wherein a source of electromagnetic beams, cells as well as nanoparticles and at least one photodetector are arranged as an optical measuring device such that electromagnetic beams excited in the nanoparticles by the electromagnetic beams of the source impinge on the photodetector, imaged thereon and as image signals can be determined quantitatively or quantitatively.

29. Device according to claim 27, wherein, in the beam path downstream of the source of electromagnetic beams and/or in the beam path in front of the photodetector, at least one beam-influencing device, at least one beam-shaping device, or at least a combination thereof is arranged.

30. Device according to claim 27, wherein the photodetector is a solid-state image sensor with photoresistors, photo diodes or photo transistors and that the solid-state image sensor is connected to a data processing system.

31. Method for detecting a substance by particle plasmon resonance (PPR) or particle-mediated fluorescence by cell surface polarization with utilization of cells, nanopartides, and at least one measuring device, comprising the method steps:
a) the surface of the cells, in which a gene whose expression leads to the polarized presentation of a protein on the surface of cells is under the control of a promoter that can be regulated by the substance to be detected, is polarized in the presence of the substance,
b) nanoparticles, functionalized with a molecule that can bind specifically to the protein that is exposed on the surface of the cells in polarized form in the presence of the substance, bind to the protein; and
c) a measurable aggregation of the nanoparticles on the surface of the cells is detected by means of at least one optical measuring device by particle plasmon resonance or particle-mediated fluorescence.

32. Method according to claim 31, wherein, as cells, cells of a first type and cells of a second type are utilized, wherein
a. the cells of the first type, in which a gene that codes for a pheromone is under the control of a promoter that can be regulated by the substance to be detected, secrete in the presence of the substance the pheromone; and
b. the surface of the cells of the second type that are responsive to the pheromone is polarized in the presence of the pheromone.

33. Method according to claim 31, wherein the method is performed by utilizing at least one device comprising:
a. cells in which a gene whose expression leads to the polarized presentation of a protein on the surface of cells is under the control of a promoter that can be regulated by the substance to be detected,
b. nanoparticles that are functionalized with a molecule that specifically binds to the surface-exposed protein,
c. at least one optical measuring device.

34. Device according to claim 28, wherein, in the beam path downstream of the source of electromagnetic beams and/or in the beam path in front of the photodetector, at least one beam-influencing device, at least one beam-shaping device, or at least a combination thereof is arranged.

35. Device according to claim 28, wherein the photodetector is a solid-state image sensor with photoresistors, photo diodes or photo transistors and that the solid-state image sensor is connected to a data processing system.
